(19) United States
(12) Patent Application Publication
(10) Pub. No.: US 2016/0158403 A1
(43) Pub. Date: Jun. 9, 2016

(54) A WOUND DRESSING

(71) Applicant: MANUKAMED LIMITED, Masterton (NZ)

(72) Inventor: Denis Eric WATSON, Masterton (NZ)

(73) Assignee: MANUKAMED LIMITED, Masterton (NZ)

(21) Appl. No.: 14/904,027

(22) PCT Filed: Jul. 9, 2014
(86) PCT No.: PCT/NZ2014/000139

§ 371 Date: Jan. 8, 2016

(30) Foreign Application Priority Data
Jul. 9, 2013 (NZ) 613058

(51) Int. Cl. A61L 15/28; A61L 15/20; A61L 15/26; A61L 15/60; A61L 15/44

(52) U.S. Cl. CPC A61L 15/28; A61L 15/60; A61L 15/44; A61L 15/26; A61L 15/20; A61L 2300/30; A61L 2300/802

(57) ABSTRACT

The present invention relates to an impregnated wound dressing having an outer membrane overlay layer and to a method of production thereof. In particular the dressing comprises a core layer that has been impregnated with a wound healing agent, such as honey, and an outer membrane overlay layer.

### FIELD OF THE INVENTION

The present invention relates to an impregnated wound dressing having an outer membrane overlay layer and to a method of production thereof. In particular the dressing comprises a core layer that has been impregnated with a wound healing agent, such as honey, and an outer membrane overlay layer.

### BACKGROUND

Wounds impact on the lives of many people. Some individuals suffer from non-healing wounds such as ulcers, infected wounds, inflamed wounds and the like. Wound dressings are needed to protect such wounds from further infection. Some wounds discharge (exude) moisture or fluids and for such wounds a wound dressing needs to be absorbent to contain the exudate while the wound dressing is in situ.

Wound dressings are often impregnated or coated with agents that facilitate wound healing or that reduce the chance of infection. Such agents include, but are not limited to, silver products, antimicrobial products such as antibiotics or honey or honey extracts or anti-inflammatory agents or products.

The main reasons for the application of a dressing are to facilitate and accelerate healing of a lesion; to prevent malodour; to minimise pain; to prevent and counteract infection; to absorb exudate and to reduce scar tissue.

Wound dressings that are impregnated with the likes of a cream or honey can be sticky or tacky in nature and can be difficult to handle when extracting the dressing from its packaging and placing on a wound. There is a need to provide an impregnated wound dressing that is dry to the touch, that does not adhere to the wound bed while still allowing a flow of exudate into the dressing, that is easy to manufacture and package and not difficult to apply to a wound.

The applicant produces a honey impregnated wound dressing that is described in PCT/GB2009/001407, the contents of which are incorporated herein in its entirety. The preferred manufacturing embodiment described in PCT/GB2009/001407 includes the step of dusting the impregnated core layer with a carboxymethylcellulose (CMC) layer. It has been found that during the dusting step involved in the manufacturing process it can be difficult to achieve a uniform thin layer of CMC dusted over the impregnated core layer.

The object of the present invention is to provide a wound dressing that overcomes the abovementioned difficulties or to at least provide the public with a useful alternative.

### SUMMARY OF THE INVENTION

In a first aspect the invention provides a wound dressing including a core layer impregnated with a wound healing agent and at least one outer membrane overlay layer.

In one embodiment the membrane overlay layer is selected from a cured carboxymethylcellulose layer, a silicone comprising layer, a polytetrafluoroethylene comprising layer, a silicone and polytetrafluoroethylene comprising layer, and a cross-linked polyurethane layer or combinations thereof.

In one embodiment the membrane overlay layer comprises a cured carboxymethylcellulose layer.

In one embodiment the carboxymethylcellulose is sodium carboxymethylcellulose.

In one embodiment the membrane overlay layer further comprises a plasticizer.

In another aspect, there is a method of producing a wound dressing as defined above, the method including the step of impregnating a core layer with a wound healing agent and then overlaying at least one surface of the impregnated core layer with a membrane overlay layer.

In one aspect there is a method of treating a wound, including the step of placing a wound dressing having a membrane overlay layer as defined above on a wound to facilitate the healing of the wound.

Additional aspects and embodiments of the invention will be apparent from the description and Figures that follow.

Definitions

The term a “membrane overlay layer” as used herein is understood to mean a pliable sheet-like structure acting as a boundary between the core layer of the wound dressing to which the membrane overlay layer is applied.

The term “ a wound healing agent” as used herein is understood to mean a liquid, cream or a gel that is impregnable into a core layer and includes, honey, gels or liquids of silver products, antimicrobial products such as antibiotics or honey or honey extracts or anti-inflammatory agents or products that promote wound healing or promote wound closure.

### BRIEF DESCRIPTION OF THE DRAWINGS

The invention will now be described by example only with reference to the FIGURE where:

FIG. 1. shows a cross-sectional view of a wound dressing of the present invention having a core layer impregnated with a wound healing agent, which core layer is sandwiched between two membrane overlay layers.

### DETAILED DESCRIPTION

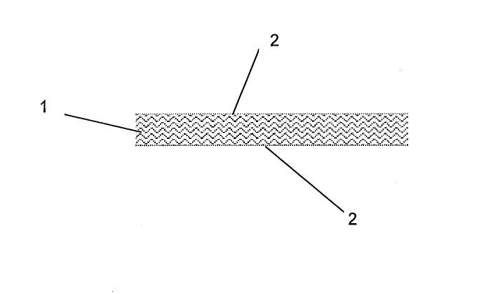

With reference to FIG. 1, in a first aspect the invention provides a wound dressing including a core layer 1 impregnated with a wound healing agent and at least one outer membrane overlay layer 2. In the embodiment illustrated the core layer 1 is sandwiched between two membrane overlay layers 2.

In one embodiment the membrane overlay layer is selected from a cured carboxymethylcellulose layer, a silicone comprising layer, a polytetrafluoroethylene comprising layer, a silicone and polytetrafluoroethylene comprising layer, and a cross-linked polyurethane layer or combinations thereof.

In one embodiment the membrane overlay layer comprises a cured carboxymethylcellulose layer. In one embodiment the carboxymethylcellulose is sodium carboxymethylcellulose.

In one embodiment the membrane overlay layer further comprises a plasticizer. In one embodiment the plasticizer is selected from glycerin, polyethylene glycol, propylene glycol, monoacetin, triacetin, triethyl citrate, sorbitol, 1,3-butanediol, D-glucono-1,5-lactone, and combinations thereof.

In one embodiment the plasticizer is glycerin.

In one embodiment the membrane overlay layer comprises a cured carboxymethylcellulose-glycerin layer.

In one embodiment the membrane overlay layer comprises a cured sodium carboxymethylcellulose-glycerin layer.

In another embodiment the membrane overlay layer comprises a blend of silicone and polytetrafluoroethylene.

In one embodiment the membrane overlay layer is between about 0.1 μm-2.0 mm in thickness. In another embodiment the overlay layer is between about 0.5 μm-1.0 mm in thickness.

In one embodiment the membrane overlay layer does not absorb any of the wound healing agent impregnated into the core layer.

In one embodiment the core layer is a superabsorbent material. In another embodiment the superabsorbent material is a superabsorbent polymer fibre, such as a cross-linked polyacrylate fiber marketed as Oasis SAF ™ or SAF™.

In one embodiment the core layer comprises an alginate. It is to be appreciated that any one of the alginate dressings in Table 1, could be impregnated with a wound healing agent, such as a honey and then coated with at least one membrane overlay layer, such as a cured carboxymethyl cellulose layer.

TABLE 1

| Manufacturer | Brand name | Dressing |
|---|---|---|
| ConvaTec | AQUACEL ® | Alginate absorbent dressing |
| ConvaTec | AQUACEL ® Ag Burn | Alginate absorbent dressing |
| ConvaTec | AQUACEL ® Extra | Two layers of Hydrofiber ® technology in |
| | | a dressing |
| 3M ™ | Tegaderm ™ | Alginate Silver absorbent dressing |
| 3M Health Care | Tegaderm ™ High gelling | Alginate wound dressing |
| | Alginate dressing | |
| 3M Health Care | Tegaderm ™ High Integrity | Alginate wound dressing |
| | Alginate dressing | |
| Derma Sciences, Inc. | AlgiCell ® | Alginate wound dressing |
| Smith & Nephew, Inc. | ALGISITE ® | Calcium alginate wound dressing |
| Coloplast Corp | Biatain ® Alginate Ag | Alginate dressing with silver |
| | Dressing | |
| Coloplast Corp | Biatain ® Soft Alginate | Alginate dressing |
| | Dressing | |
| McKesson MedicalSurgical | | Calcium Alginate Rope Dressing |
| | | Calcium Alginate Sheet Dressing |
| DermaRite Industries, LLC | DermaGinate ™ 12″ Rope | |
| | DermaGinate ™ Dressing | |
| MPM Medical, Inc | ExcelGinate ™ | Non-woven calcium alginate dressing |
| Gentell Wound and Skin Care | Gentell ® Calcium | Calcium alginate dressing |
| | Alginate | |
| DeRoyal | Kalginate ™ | Heavy-fiber alginate dressing |
| ConvaTec | KALTOSTAT ® | Absorbent gel-fiber |
| | KALTOSTAT ® Rope | |
| Covidien | Kendall TM Calcium | Calcium alginate dressing |
| | Alginate Dressing | |
| Covidien | Kendall TM Calcium-Zinc | Calcium-Zinc alginate dressing |
| | Alginate Dressing | |
| Medline Industries, Inc. | Maxorb ® II | 100% alginate dressing |
| Medline Industries, Inc. | Maxorb ® Extra Calcium | calcium alginate and sodium |
| | Alginate | carboxymethylcellulose fibers |
| Medline Industries, Inc. | Opticell | Alginate dressing |
| MediPurpose ®, Inc. | MediPlusTM Alginate | a calcium containing dressing derived |
| | Dressing | from seaweed |
| Mölnlycke Health Care US, | Melgisorb ® Calcium | A calcium alginate dressing |
| LLC | Alginate Dressing | |
| Hollister Wound Care | Restore ® Calcium | Calcium alginate dressing |
| | Alginate Dressing | |
| Hartmann USA, Inc. | Sorbalgon ® | An absorbent calcium alginate dressing |
| Mylan Bertek | Sorbsan ® | A calcium alginate dressing |
| Pharmaceuticals, Inc. | | |

It is also to be appreciated that the membrane overlay layer may comprise a silicone comprising layer, a polytetrafluoroethylene comprising layer, a silicone and polytetrafluoroethylene comprising layer, a cross-linked polyurethane layer or combinations thereof. The membrane overlay layer is preferably non-adherent and sterilizable for wound dressing applications. In some instances it would be appropriate to fenestrate or perforate the membrane overlay layer to allow for release of the wound healing agent from the core layer into the wound and the flow of exudate through the membrane overlay layer in contact with the wound bed, when in use. Such fenestration or perforation would take place in the wound dressing manufacturing process once the membrane overlay layer was in situ over the impregnated core layer. An example of a product that would also be suitable for use as an overlay layer is Silon™, which is produced commercially by BioMed Sciences. Silon™ comprises a blend of silicone and polytetrafluoroethylene. Silon™ can be readily fenestrated, perforated or sliced to interrupt the integrity of the membrane layer.

In one embodiment the wound healing agent is selected from a honey, a silver agent, an antimicrobial agent or mixtures thereof.

In one embodiment the wound healing agent is a honey derived from the Leptospermum genus. In one embodiment the wound healing agent is a honey derived from Leptospermum scoparium.

In another aspect, there is a method of producing a wound dressing as defined above, the method including the step of impregnating a core layer with a wound healing agent and then overlaying at least one surface of the impregnated core layer with a membrane overlay layer.

In one embodiment the method includes the step of packaging the wound dressing between protective liners.

In a further embodiment the method includes the step of packaging the wound dressing into a sealable pouch package.

In one embodiment the method includes the further step of sterilizing the dressing. In one embodiment the sterilization step is achieved by gamma irradiation.

In one aspect there is a method of treating a wound, including the step of placing a wound dressing as defined above on a wound to facilitate the healing of the wound.

The examples described herein are provided for the purpose of illustrating specific embodiments of the invention and are not intended to limit the invention in any way. It is understood that variations and modifications may be made without departing from the scope of the invention. Persons of ordinary skill can utilize the disclosures and teachings herein to produce other embodiments and variations without undue experimentation. Furthermore, where known equivalents exist to specific features, such equivalents are incorporated as if specifically referred to in this specification. All such embodiments, variations, and equivalents are considered to be part of this invention.

### EXAMPLES—MANUFACTURE OF THE DRESSING

### Example 1

Core Layer is OASIS SAF® impregnated with Leptospermum derived honey.

As described in PCT/GB2009/001407, the manufacture of OASIS SAF® by Technical Absorbents comprises polymerisation in water followed by extrusion of the aqueous polymer solution in a hot air stream to dry and cure the polymer, thereby producing insoluble polymer fibres. An extremely high conversion rate of the raw materials to polymer is achieved. Moisture may be added to the fibres to aid processing, and the fibres are precision cut into a range of staple lengths. The OASIS Super Absorbent technology can also be used to produce filament yarns (OASIS-FIL) and polymer solutions (OASIS-PS), either of which may be used in the invention. Typically, the thickness of the SAF layer when in the form of a sheet ranges from 0.25 mm to 10 mm; for example, the sheet may be 0.5 mm, 1 mm, 1.5 mm, 2 mm, 2.5 mm, 3 mm, 3. 5mm, 4 mm, 4.5 mm, 5 mm, 5.5 mm, 6 mm, 6.5 mm, 7 mm, 7.5 mm, 8mm, 8.5 mm, 9 mm, 9.5 mm or 10 mm thick. Preferably, the sheet is less than 5 mm, 4 mm or 3 mm thick and more preferably less than 2 mm thick.

The OASIS SAF fibre product is then impregnated with honey. The honey may be impregnated by continuous (roll to roll) dip coating. In this method, a roll of the super-absorbent sheet pre-cut to a desired width is drawn through an immersion tank of warm honey (typically between 35° C.-50° C., for example 35° C.-45° C.), typically at a constant speed after which it is wound into a roll. It is appreciated that the sheet to be impregnated need only be just beneath the surface in the tank of honey. Blades to remove excess honey are combined with a nip roller to create a constant pressure at the point of exit from the tank. The degree of impregnation of the sheet with the honey is determined by the submersion time and the time under roller pressure. It is therefore important to regulate the honey level within the tank and the time under roller pressure. The level of honey in the tank is maintained by pumping more honey into the bath when the level is reduced. Typical dwell times in the honey tank are in the region of 2-6 seconds depending on the material being impregnated. The speed is determined by the tensile strength along the length of the material. After leaving the tank and passing the nip rollers the impregnated sheet is dried before being wound into a roll form. The roll is further processed into cut lengths for use as a wound dressing. It will be appreciated that other methods of impregnation may also be used such as, for example, batch process of emersion.

The superabsorbent material is preferably impregnated with the honey such that the honey does not saturate the super-absorbent material, thereby leaving capacity for absorbing lesion fluid components. In any event, it is appreciated that the super-absorbent material is impregnated with the honey such that the honey does not utilise all of the super-absorbent material's capacity to absorb fluid. It is believed that the honey is held by the superabsorbent fibre material by way of capillary action. The pH of honey is typically around 3.9-4.3 and the applicant believes that the absorbent capacity of the superabsorbent fibres at the typical pH of honey is shut down and that the fibres do not absorb any of the free water in honey. While it is believed that the honey coats the super-absorbent material (eg fibres) and reduces the absorbency rate of the fibres it is further believed that as the honey is diluted by wound exudate, the fibres' capacity for absorption is restored, possibly because the pH of the honey increases as it is diluted.

Preferably, the super-absorbent material is impregnated with honey such that the super-absorbent material is able to absorb at least 10 times its dry weight when in water, and more preferably at least 20, 30, 40 or 50 times its dry weight. For example, when the super-absorbent material is impregnated with honey in a weight ratio of 4:1, the super-absorbent material typically absorbs 10 times its dry weight when in water.

The ability of a super-absorbent material to hold honey within its structure is dependent on the mass, density and type of the absorbent material used and the impregnation method used. A typical dosage of honey which does not lead to saturation is 0.2 g/cm2, and so for a 5 cm×5 cm sheet the dosage would be 5 g honey and for a 10 cm×10 cm sheet the dosage would be 20 g honey. Thus the sheet may contain between 0.1 g/cm² and 3.0 g/cm2 such as between 0.15 g/cm² and 3 g/cm². However, it will be appreciated that other dosages outside this range may be used which give the required utilisation of absorbent capacity, depending upon the super-absorbent material used and the thickness of the sheet.

The membrane overlay layer is prepared by taking a polyester (PET) carrier film and coating the film with a layer of sodium carboxymethylcellulose (NaCMC) (99-90%) -and glycerin (up to 10%) Suitable sodium CMC is commercially available from Hercules, Inc. (Wilmington, Del.) under the AQUALON brand.

The coating is achieved by spraying the NaCMC/glycerin mixture onto the

PET carrier film. It is to be appreciated that other coating systems may be used for example the Meyer-Bar technique. The PET film is then used to “carry” the NaCMC-glycerin layer as the PET film and NaCMC-glycerin layer are infrared radiated to cure the NaCMC-glycerin layer to form a polymeric layer and to create a CMC cured film that can be subsequently removed from the PET carrier film. The separated NaCMC-glycerin cured layer can then be rolled onto at least one surface of the honey impregnated OASIS SAF core layer described above to prepare a roll of wound dressing of the invention. The roll of wound dressing can then be cut to produce the desired shape and size of the wound dressings. The wound dressings can then be placed between liner layers and pouched to produce a single wound dressing. The wound dressing would then be sterilized by a process such as by way of gamma irradiation.

### Example 2: Core Layer is OASIS SAF® impregnated with Leptospermum derived honey and having a Silon™ coating.

The core layer of Oasis SAF was prepared and impregnated with Leptospermum derived honey.

A membrane overlay layer was prepared by BioMed Sciences and then applied by rolling the Silon™ layer onto at least one surface of the honey impregnated OASIS SAF core layer described above to prepare a roll of wound dressing of the invention. Silon™ is an example of a combination product comprising a blend of both silicone and polytetrafluoroethylene (PTFE) also known as Teflon™. The wound dressing was then fenestrated by feeding the dressing through a cutting tool to slit the Silon™ layer and to partially slit the core OASIS SAF layer. It is to be appreciated that other means of perforating the membrane layer would also be possible, such as passing the dressing by a needle press that presses a plurality of needles through the dressing to perforate the outer membrane layer. The wound dressing is then cut to produce the desired shape and size of the wound dressings. The wound dressing may then be optionally placed between liner layers and then pouched to produce a single wound dressing. The wound dressing would then be sterilized by a process such as by way of gamma irradiation.

The present invention and its embodiments have been described in detail. However, the scope of the present invention is not intended to be limited to the particular embodiments of the invention described in the specification. Various modifications, substitutions, and variations can be made to the disclosed material without departing from the essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention.

1. A wound dressing including a core layer impregnated with a wound healing agent and at least one outer membrane overlay layer.

2. The wound dressing as claimed in claim 1 wherein the overlay membrane layer is selected from a cured carboxymethylcellulose layer, a silicone comprising layer, a polytetrafluoroethylene comprising layer, a silicone and polytetrafluoroethylene comprising layer, and a cross-linked polyurethane layer or combinations thereof.

3. The wound dressing as claimed in claim 1 wherein the membrane overlay layer comprises a cured carboxymethylcellulose layer.

4. The wound dressing as claimed in claim 2 wherein the carboxymethylcellulose is sodium carboxymethylcellulose.

5. The wound dressing as claimed in claim 1 wherein the membrane overlay layer comprises a silicone and polytetrafluoroethylene blend layer.

6. The wound dressing as claimed in claims 1 wherein the wound healing agent is selected from a silver comprising agent or a honey comprising agent.

7. The wound dressing as claimed in claim 6 wherein the wound healing agent comprises honey derived from the Leptospermum species.

8. The wound dressing as claimed in claim 7 wherein the wound healing agent comprises a honey derived from Leptospermum scoparium.

9. The wound dressing as claimed in any one of claim 1 wherein the membrane overlay layer further comprises a plasticizer.

10. The wound dressing as claimed in claim 9 wherein the plasticizer is selected from glycerin, polyethylene glycol, propylene glycol, monoacetin, triacetin, triethyl citrate, sorbitol, 1,3-butanediol, D-glucono-1,5-lactone, and combinations thereof.

11. The wound dressing as claimed in claim 10 wherein the plasticizer is glycerin.

12. The wound dressing as claimed in claim 1 wherein the core layer is selected from an alginate or a superabsorbent material.

13. The wound dressing as claimed in claim 1 wherein the core layer comprises a polyacrylate superabsorbent polymer fiber.

14. The wound dressing as claimed in claim 1 wherein the membrane overlay layer does not absorb any of the wound healing agent impregnated into the core layer.

15. A method of producing a wound dressing as claimed in claim 1, the method including the step of impregnating a core layer with a wound healing agent and then overlaying at least one surface of the impregnated core layer with an overlay layer.

16. The method as claimed in claim 15 wherein the method includes the further step of fenestration or penetration of the membrane overlay layer.

17. A method of treating a wound, including the step of placing a wound dressing as claimed in claim 1 onto a wound to facilitate the healing of the wound.
